(19) United States
(12) Patent Application Publication
(10) Pub. No.: US 2019/0099096 A1
(43) Pub. Date: Apr. 4, 2019

(54) DISPLAY DEVICE AND METHOD OF DETECTING HEART RATE INFORMATION

(71) Applicant: BOE Technology Group Co., Ltd., Beijing (CN)

(72) Inventors: Pengpeng Wang, Beijing (CN); Haisheng Wang, Beijing (CN); Xiaoliang Ding, Beijing (CN); ChihJen Cheng, Beijing (CN)

(21) Appl. No.: 16/140,005

(22) Filed: Sep. 24, 2018

(30) Foreign Application Priority Data
Sep. 29, 2017 (CN) 201710911250.4

(51) Int. Cl. A61B 5/024; A61B 5/0245; H01L 51/52; H01L 27/32; G03F 7/09

(52) U.S. Cl. CPC A61B 5/02405; A61B 5/0245; G03F 7/09; H01L 27/3281; H01L 51/5237; H01L 51/5284

(57) ABSTRACT

A display device is provided. The display device includes a base substrate; an encapsulation layer, on the base substrate; an organic light-emitting display element between the base substrate and the encapsulation layer configured to emit a first light passing though the base substrate and a second light passing through the encapsulation layer to display image; a photosensitive layer between the base substrate and the encapsulation layer configured to detect a light reflected from a skin of a user and generate a photocurrent signal; and a photosensitive detection circuit coupled to the photosensitive layer configured to determine heart rate information according to the photocurrent signal generated by the photosensitive layer and output the heart rate information.

### CROSS-REFERENCES TO RELATED APPLICATIONS

This application claims priority to Chinese patent application No. 201710911250.4 filed on Sep. 29, 2017, which is incorporated herein by reference in its entirety.

### FIELD

The present disclosure relates to the field of display technologies, and particularly to a display device and a method of detecting heart rate information.

### BACKGROUND

In recent years, with the development of the sciences and technologies, the Internet of Things era when things are interconnected is coming. In the Internet of Things. Data required for things interconnecting in the architecture of the Internet of Things should be acquired using various sensors, so cheap, highly integrated, and easy-to-use sensors are expected to be popular in future.

Mobile devices, e.g., mobile phones, etc., are so popularized that all of us have been focused on our screens, and the screens are indispensable to our life. As per Ockham's Razor, a screen being integrated together with various sensors, operating as information input and output terminals, may be an ultimate modality of future scientific and technological products, so future scientific and technological efforts is focused on integration of various sensors with a screen.

### SUMMARY

In an aspect, an embodiment of the disclosure provides a display device. The display device includes a base substrate, an encapsulation layer, on the base substrate; an organic light-emitting display element between the base substrate and the encapsulation layer configured to emit a first light passing though the base substrate and a second light passing through the encapsulation layer to display image; a photosensitive layer between the base substrate and the encapsulation layer configured to detect a light reflected from a skin of a user and generate a photocurrent signal; and a photosensitive detection circuit coupled to the photosensitive layer configured to determine heart rate information according to the photocurrent signal generated by the photosensitive layer and output the heart rate information.

In a possible implementation, in the display device above according to the embodiment of the disclosure, the display device further includes a light-shielding layer located on a side of the photosensitive layer away from the base substrate, wherein a positive projection of the light-shielding layer onto the base substrate overlaps with a positive projection of the photosensitive layer onto the base substrate.

In a possible implementation, in the display device above according to the embodiment of the disclosure, the organic light-emitting display element includes a first electrode layer, a second electrode layer located on a side of the first electrode layer close to the base substrate, and an organic functional layer located between the first electrode layer and the second electrode layer, wherein the first electrode layer and the second electrode layer are made of a transparent electrically-conductive material.

In a possible implementation, in the display device above according to the embodiment of the disclosure, the photosensitive layer includes at least one photosensitive detection electrode coupled to the photosensitive detection circuit, the at least one photosensitive detection electrode is configured to detect the light reflected from the skin of the user and generate the photocurrent signal.

In a possible implementation, in the display device above according to the embodiment of the disclosure, the first electrode layer includes a plurality of first electrodes extending along a first direction; the second electrode layer includes a plurality of second electrodes extending along a second direction intersecting with the first direction; the organic functional layer includes light-emitting elements at positions where the first electrodes intersect with the second electrodes; and a positive projection of the at least one photosensitive detection electrode does not fully overlap with positive projections of the light-emitting elements onto the base substrate.

In a possible implementation, in the display device above according to the embodiment of the disclosure, the photosensitive layer includes a plurality of photosensitive detection electrodes distributed in an array, wherein positive projections of the photosensitive detection electrodes onto the base substrate do not overlap with any of positive projections of the first electrodes, the second electrodes, and the light-emitting elements onto the base substrate.

In a possible implementation, in the display device above according to the embodiment of the disclosure, the photosensitive layer includes a plurality of strip-shaped photosensitive detection electrodes arranged in parallel, wherein the photosensitive detection electrodes extend along the first direction, and positive projections of the photosensitive detection electrodes and positive projections of the first electrodes onto the base substrate do not overlap with each other; or the photosensitive detection electrodes extend along the second direction, and the positive projections of the photosensitive detection electrodes and positive projections of the second electrodes onto the base substrate do not overlap with each other.

In a possible implementation, in the display device above according to the embodiment of the disclosure, the photosensitive layer includes a photosensitive detection electrode arranged as an integral plane, wherein the photosensitive detection electrode is provided with hollow areas at its portions overlapping with the light-emitting elements.

In a possible implementation, in the display device above according to the embodiment of the disclosure, the photosensitive detection circuit includes at least one driver signal line extending along the first direction, at least one detection signal line extending along the second direction, and at least one switch transistor, wherein the switch transistor has a gate connected with the driver signal line, a source connected with the photosensitive detection electrode, and a drain connected with the detection signal line.

In a possible implementation, in the display device above according to the embodiment of the disclosure, the photosensitive detection circuit further includes a current-voltage conversion circuit, a filter circuit connected with the current-voltage conversion circuit, an amplifying circuit connected with the filter circuit, a counter connected with the amplifying circuit, and a timer connected with the counter, wherein the current-voltage conversion circuit is connected with the at least one detection signal line, and configured to convert the photocurrent signal on the at least one detection signal line into a voltage signal; the filter circuit is configured to filter the voltage signal; the amplification circuit is configured to amplify the filtered voltage signal; and the counter is configured to count under the control of the timer to obtain a value of a heart rate.

In a possible implementation, in the display device above according to the embodiment of the disclosure, the at least one driver signal line is further connected with the organic light-emitting display element to drive the organic light-emitting display element to display the image in a display period, and to drive the photosensitive detection electrodes to generate the photocurrent signal and transmit the photocurrent signal to the photosensitive detection circuit in a heart rate detection period.

In a possible implementation, in the display device above according to the embodiment of the disclosure, the display device further includes a black matrix layer located on a side of the organic light-emitting display element away from the base substrate; the black matrix layer covers a portion of the organic light-emitting display element, and covers at least one photosensitive detection electrode; and the photosensitive detection circuit further includes a mode switching circuit, wherein the mode switching circuit is configured, in an active light mode, to control the photosensitive detection electrodes in an area covered by the black matrix layer to detect the first light reflected from the skin and generate the photocurrent signal, and in an ambient light mode, to control the photosensitive detection electrodes out of the area covered by the black matrix layer to detect an ambient light reflected from the skin and generate the photocurrent signal in an ambient light mode.

In a possible implementation, in the display device above according to the embodiment of the disclosure, the active light mode is a period when the organic light-emitting display element is displaying an image or sleeping, and the ambient light mode is a period when the organic light-emitting display element is disabled.

In a possible implementation, in the display device above according to the embodiment of the disclosure, the photosensitive layer is configured to detect the first light and/or an ambient light reflected from the skin of the user, and generate the photocurrent signal.

In a possible implementation, in the display device above according to the embodiment of the disclosure, the photosensitive layer is located on a side of the organic light-emitting display element close to the base substrate.

In a possible implementation, in the display device above according to the embodiment of the disclosure, the display device further includes a line layer located between the base substrate and the organic light-emitting display element, wherein the photosensitive detection circuit is arranged at same layer as the line layer; and the photosensitive layer is located between the organic light-emitting display element and the line layer.

In a possible implementation, the display device above according to the embodiment of the disclosure is a wearable display device.

In a possible implementation, in the display device above according to the embodiment of the disclosure, the photosensitive detection electrode is made of a PIN-type semiconductor material or a PN-type semiconductor material.

In another aspect, an embodiment of the disclosure provides a method of detecting heart rate information by the display device above. The method includes: emitting, by the organic light-emitting display element, a first light passing through the base substrate, emitting a second light passing through the encapsulation layer; detecting, by the photosensitive layer, a light reflected from a skin of a user and generating a photocurrent signal; and determining, by the photosensitive detection circuit, the heart rate information according to the photocurrent signal generated by the photosensitive layer.

In another aspect, in the method above according to the embodiment of the disclosure, detecting, by the photosensitive layer, the light reflected from the skin of the user and generating the photocurrent signal includes: detecting, by the photosensitive layer, the first light and/or ambient light reflected from the skin of the user, and generating the photocurrent signal.

### BRIEF DESCRIPTION OF THE DRAWINGS

FIG. 1 is a schematic structural diagram of a display device according to an embodiment of the disclosure;

FIG. 2A to FIG. 2D are top views of the display device according to the embodiment of the disclosure;

FIG. 3 is a partially enlarged diagram of a photosensitive detection electrode in FIG. 2A;

FIG. 4 is a schematic structural diagram of a photosensitive detection circuit according to an embodiment of the disclosure;

FIG. 5 is another schematic structural diagram of a display device according to an embodiment of the disclosure;

FIG. 6 is another top view of the display device according to the embodiment of the disclosure; and

FIG. 7 is a flow chart of a method of detecting heart rate information according to an embodiment of the disclosure.

### DETAILED DESCRIPTION

Implementations of the display device according to the embodiment of the disclosure will be described below in details with reference to the drawings. The thicknesses and shapes of respective layers in the drawings are not intended to reflect any real proportion, but only intended to illustrate the content of the disclosure.

A mobile display device with a heart rate detection function has come into productization, but this product is formed by combining a display screen module with a heart rate detection sensor module. The mobile display device, formed by combining the display screen module with a heart rate detection sensor module, includes a display screen, an optical sensor, a light source of the optical sensor, a processor (for example CPU), etc., so the mobile display device with the heart rate detection function in the related art suffers from a large volume, a large thickness, a high weight, a high cost, etc.

In view of the problems of a large volume, a large thickness, a high weight, a high cost, etc., of the mobile display device with the heart rate detection function, an embodiment of the disclosure provides a display device.

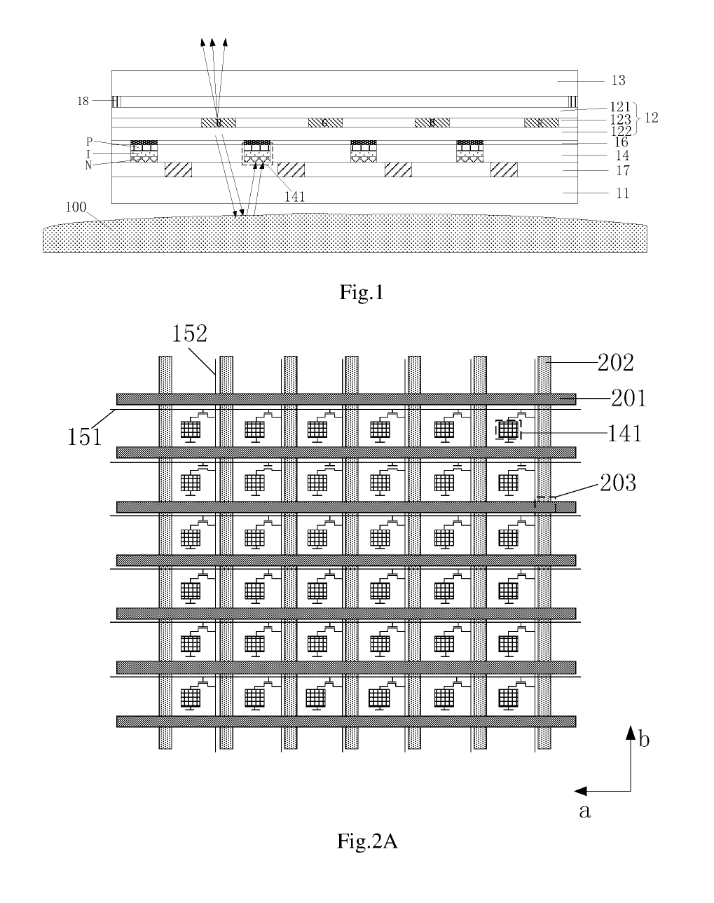

As illustrated in FIG. 1, an embodiment of the disclosure provides a display device. The display device includes a base substrate 11, an encapsulation layer 13 on the base substrate 11, an organic light-emitting display element 12 between the base substrate 11 and the encapsulation layer 13; a photosensitive layer 14 located between the base substrate 11 and the encapsulation layer 13, and a photosensitive detection circuit coupled to the photosensitive layer 14.

The organic light-emitting display element 12 is configured to emit a first light passing though the base substrate 11 and a second light passing through the encapsulation layer 13 to display image.

The photosensitive layer 14 is configured to detect light reflected from a skin and generate a photocurrent signal.

The photosensitive detection circuit is configured to determine heart rate information according to the photocurrent signal generated by the photosensitive layer 14 and output the heart rate information.

In the display device according to the embodiment of the disclosure, the organic light-emitting display element 12 can emit light bidirectionally, and the photosensitive layer 14 is arranged between the base substrate 11 and the encapsulation layer 13, so that the photosensitive layer 14 can use the light emitted from the organic light-emitting display element 12 or ambient light as a light source, without arranging light source separately, thus reducing the size of a heart rate detection element, and lowering power consumption of heart rate detection.

In some embodiments, the photosensitive layer 14 includes at least one photosensitive detection electrode 141 coupled to the photosensitive detection circuit and configured to detect the light reflected from the skin of the user and generate the photocurrent signal.

In some embodiments, the photosensitive layer 14 is configured to detect the first light reflected from the skin of the user, and/or an ambient light reflected from the skin of the user and generate the photocurrent signal.

In a real application, referring to FIG. 1, the first light emitted from an organic light-emitting display element 12 (or ambient light) is transmitted to the skin 100, the first light or the ambient light is transmitted through various tissues and blood in the skin and reflected back, the reflected light is received by at least one photosensitive detection electrode 141. The light transmitted to the skin 100 may be absorbed by the various tissues and the blood in the skin, where the amount of light absorbed by the various tissues in the skin is substantially constant, and the blood flow varies as the beating of the heart, so the amount of light absorbed by the blood also varies. While the heart is beating, the blood flow increases, and the amount of light absorbed by the blood also increases; and during a break between beats of the heat, the blood flow decreases, and the amount of light absorbed by the blood also decreases, so the photosensitive detection circuit can determine the value of a heart rate according to the photocurrent signal generated by the photosensitive detection electrode 141 detecting light.

In some embodiments, the base substrate 11 and the encapsulation layer 13 are made of a light-transmitting material, and optionally a transparent material, e.g., glass, etc., or can be made of a material with some light transmittivity as long as light can be transmitted, although the material of the base substrate 11 and the encapsulation layer 13 will not be limited thereto. Furthermore the second light emitted from the organic light-emitting display element 12 passes through the encapsulation layer 13 to display image, and the first light passes through the base substrate 11, that is, organic light-emitting display element 12 can emit light bidirectionally. Since light is also transmitted through the organic light-emitting display element 12, the photosensitive layer 14 uses the first light emitted from the organic light-emitting display element 12 as a light source for heart rate detection, or uses ambient light as a light source for heart rate detection, without arranging light source separately, thus lowering power consumption of heart rate detection. Furthermore only the photosensitive layer 14 and the photosensitive detection circuit are added, where the photosensitive layer 14 is a layer between any two thin layers between the base substrate 11 and the encapsulation layer 1, and the photosensitive detection circuit can be a layer arranged between the base substrate 11 and the encapsulation layer 13, or the photosensitive detection circuit can be integrated into some layer between the base substrate 11 and the encapsulation layer 13. For example, the photosensitive detection circuit can be integrated with a driver circuit of the organic light-emitting display element 12, so the volume of the display device with the heart rate detection function according to this embodiment can substantially remain as it is except for a slightly larger thickness thereof, and as compared with the addition of a specific light source (e.g., an LED lamp), the display device according to the disclosure can significantly reduce the volume of a mobile display device with a heart rate detection function, so the display device according to the embodiment of the disclosure is advantageous in a small volume, low power consumption, a low weight, a small thickness, etc.

Since the display device according to the embodiment of the disclosure is advantageous in a small volume, low power consumption, a low weight, a small thickness, etc., in some embodiments of the disclosure, the display device is a wearable display device, and it is applicable to a wearable product, e.g., a wrist ring, a wrist watch, or another device with a physical sign detection function.

In some embodiments, the organic light-emitting display element 12 is Passive Matrix OLED (PMOLED). Referring to FIG. 1, reference numeral 17 in FIG. 1 refers to the line layer of the display device, where the line layer includes the driver circuit configured to drive the light-emitting elements to emit light. Since no pixel compensation circuit is required for the pixel of the PMOLED element, the driver circuit driving the PMOLED element to display is simple, so the photosensitive detection circuit above can be integrated into the driver circuit without increasing the volume of the display device.

Furthermore in the display device above according to the embodiment of the disclosure, referring to FIG. 1, the display device further includes: a light-shielding layer 16 located on the side of the photosensitive layer 14 away from the base substrate 11.

A positive projection of the light-shielding layer 16 onto the base substrate overlaps with a positive projection of the photosensitive layer 14 onto the base substrate.

The light-shielding layer 16 is located on the side of the photosensitive layer 14 away from the base substrate 11, and the positive projection of the light-shielding layer 16 onto the base substrate overlaps with the positive projection of the photosensitive layer 14 onto the base substrate, so the light-shielding layer 16 shields the light transmitted to the photosensitive detection electrodes 141 from above, e.g., the first light transmitted to the photosensitive detection electrodes 141 from the light-emitting element above, or the light-shielding layer 16 shields the ambient light transmitted to the photosensitive detection electrodes 141 from above, to avoid the light above from being absorbed by the photosensitive detection electrodes 141, which would otherwise have affected a detection result. The light-shielding layer 16 may be arranged in one implementation of the disclosure, but the light-shielding layer 16 may not be arranged in another implementation of the disclosure, and before a heart rate is detected, the photosensitive detection electrodes 141 can detect the strength of light incident perpendicularly thereto, and calculate the heart rate by subtracting the strength of the light incident perpendicularly thereto, thus alleviating heart rate detection from being affected by this portion of the light.

In some embodiments of the disclosure, in the display device, as illustrated in FIG. 1, the organic light-emitting display element 12 includes: a first electrode layer 121, a second electrode layer 122 located on the side of the first electrode layer 121 close to the base substrate 11, and an organic functional layer 123 located between the first electrode layer 121 and the second electrode layer 122.

The first electrode layer 121 and the second electrode layer 122 are made of a transparent electrically-conductive material.

In some embodiments of the disclosure, the first electrode layer 121 is arranged as an anode, and the second electrode layer 122 is arranged as a cathode, or the first electrode layer 121 is arranged as a cathode, and the second electrode layer 122 is arranged as an anode. The first electrode layer 121 and the second electrode layer 122 are made of a transparent electrically-conductive material, so the organic light-emitting display element 12 can emit light bidirectionally.

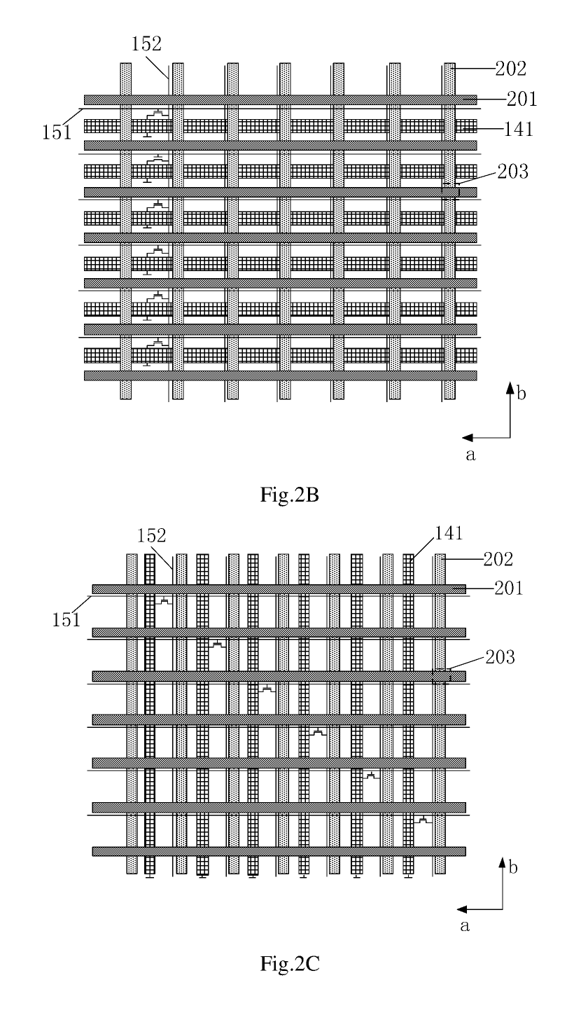

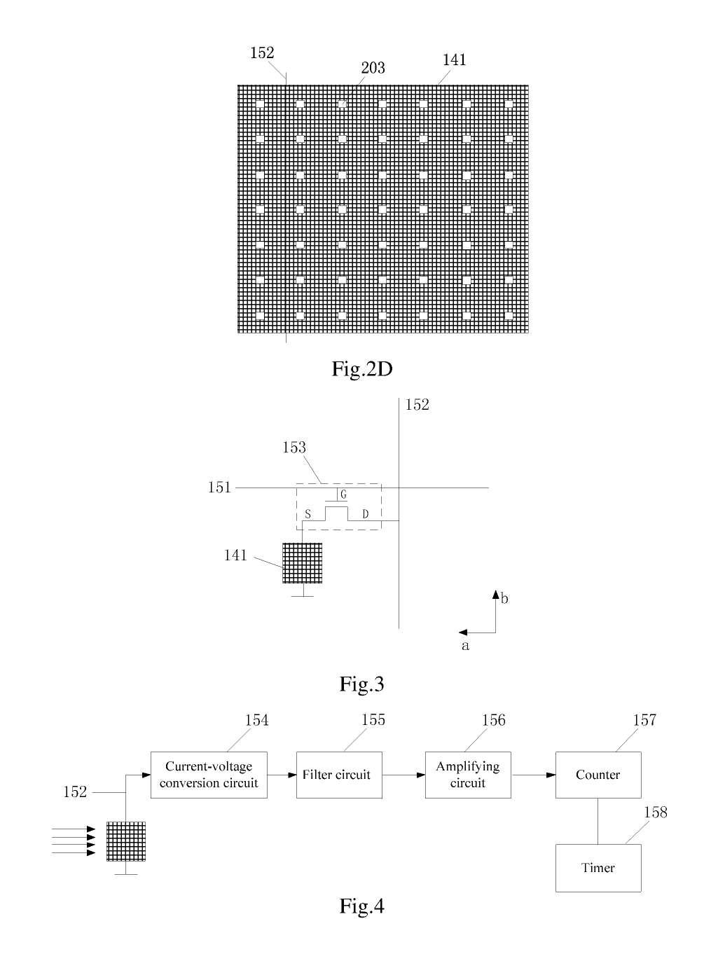

In some embodiments of the disclosure, in the display device, as illustrated in FIG. 2A, the first electrode layer 121 includes a plurality of first electrodes 201 extending along a first direction (i.e., the direction as denoted by the arrow a as illustrated).

The second electrode layer 122 includes a plurality of second electrodes 202 extending along a second direction intersecting with the first direction (i.e., the direction as denoted by the arrow b as illustrated).

The organic functional layer 123 includes light-emitting elements 103 at the positions where the first electrodes 201 intersect with the second electrodes 202.

Positive projections of the photosensitive detection electrodes 141 and the positive projections of the light-emitting elements 203 onto the base substrate 11 do not fully overlap with each other.

Referring to FIG. 1 again, the first light emitted from a light-emitting element 203 (e.g., the pixel R in FIG. 1) are incident obliquely on the surface of the skin, reflected by the surface, and then received by the photosensitive detection electrode 141. When the positive projection of the light-emitting element 203 and the photosensitive detection electrode 141 onto the base substrate 11 fully overlap with each other, that is, the light-emitting element 203 is located right above the photosensitive detection electrode 141, then the photosensitive detection electrode 141 shields the first light emitted downward from the light-emitting element 203, so less first light emitted from the light-emitting element 203 is transmitted to the skin, that is, the strength of the reflected light of the skin, which can be detected by the photosensitive detection electrode 141 is very low, thus affecting the result of detecting a heart rate. Accordingly in some embodiments of the disclosure, the positive projection of the photosensitive detection electrode 141 and the positive projection of the light-emitting element 203 onto the base substrate 11 do not fully overlap with each other.

The light-emitting element 203 is located at the position where a first electrode 201 intersects with a second electrode 202, and after a signal is applied to the first electrode 201 and the second electrode 202, a pixel element at the position of the intersection emits light at a corresponding strength as a function of the strength of the signal.

In a specific implementation, the photosensitive detection electrodes 141 at the photosensitive layer 14 are distributed in various patterns, which will be described below in details with reference to the drawings.

One Pattern

As illustrated in FIG. 2A, the photosensitive layer 14 includes a plurality of photosensitive detection electrodes 141 distributed in an array.

Positive projections of the photosensitive detection electrodes 141 onto the base substrate 11 do not overlap with any of positive projections of the first electrodes 201, the second electrodes 202, and the light-emitting elements 203 onto the base substrate 11.

Referring to FIG. 2A, the positive projections of the photosensitive detection electrodes 141 onto the base substrate 11 do not overlap with any of the positive projections of the first electrodes 201, the second electrodes 202, and the light-emitting elements 203 onto the base substrate 11, that is, the photosensitive detection electrodes 141 are located at the gaps between the first electrodes 201 and the second electrodes 202, so that the positive projections of the photosensitive detection electrodes 141 and the positive projections of the light-emitting elements 203 onto the base substrate 11 do not overlap with each other, the first light emitted from the light-emitting elements 203 can be transmitted to the skin as many as possible, and thus the strength of the first light, reflected by the skin, and received by the photosensitive detection electrodes can be higher, thus resulting in a more accurate detection result.

The plurality of photosensitive detection electrodes 141 are arranged to be distributed in an array as illustrated in FIG. 2A, so that in a specific detection process, signal lines for driving the organic light-emitting display elements 12 to display are reused for heart rate detection by the photosensitive detection electrodes 141, to thereby reduce the number of wires, and lower a cost of the display device.

In a specific implementation, the photosensitive detection electrodes 141 are arranged in various shapes, e.g., a square, a round, an ellipse, a polygon, etc., although the shape of the photosensitive detection electrodes 141 will not be limited thereto.

Another Pattern

As illustrated in FIG. 2B, the photosensitive layer 14 includes a plurality of strip-shaped photosensitive detection electrodes 141 arranged in parallel.

The photosensitive detection electrodes 141 extend along the first direction, and positive projections of the photosensitive detection electrodes 141 and positive projections of the first electrodes 201 onto the base substrate 11 do not overlap with each other, and as illustrated in FIG. 2B, the photosensitive detection electrodes 141 each is located between two adjacent first electrodes 201.

Alternatively, the photosensitive detection electrodes 141 extend along the second direction, and positive projections of the photosensitive detection electrodes 141 and positive projections of the second electrodes 202 onto the base substrate 11 do not overlap with each other, and as illustrated in FIG. 2C, the photosensitive detection electrodes 141 each is located between two adjacent second electrodes 202.

The photosensitive detection electrode 141 is arranged in a strip shape, so a photosensitive detection electrode 141 receives more light, so the strength of a photocurrent signal generated by the photosensitive detection electrode 414 is higher, and thus a detection result is subject to less interference of noise or another factor, thus resulting in a more accurate detection result.

Another Pattern

As illustrated in FIG. 2D, the photosensitive layer 14 includes a photosensitive detection electrode 141 arranged as an integral plane.

The photosensitive detection electrode 141 is provided with hollow areas at its portions of which the positive projections overlap with the light-emitting elements 203.

The photosensitive detection electrode 141 is arranged as an integral plane, and the photosensitive detection electrode 141 is provided with hollow areas at its portions overlapping with the light-emitting elements 203, so that the photosensitive layer 14 receives the largest amount of light, that is, photosensitive detection is performed in the largest area, so the strength of a photocurrent signal generated by a photosensitive detection electrode 141 is the highest, thus facilitating reading of a heart rate signal.

FIG. 3 is a partially enlarged diagram of a photosensitive detection electrode 141 in FIG. 2A. Referring to FIG. 3, the photosensitive detection circuit includes: driver signal lines 151 extending along the first direction, detection signal lines 152 extending along the second direction, and switch transistors.

The switch transistor 153 each has a gate G connected with the driver signal line 151, a source S connected with the photosensitive detection electrode 141, and a drain D connected with the detection signal line 152.

When detecting a signal, a driver signal line 151 applies a voltage to the gate G of a switch transistor 153, so that the source S and the drain D of the switch transistor 153 are connected, so a detection signal line 152 reads a photocurrent signal generated by a photosensitive detection electrode 141.

Further referring to FIG. 2A, in the structure of the photosensitive layer 14 in the one pattern above, when detecting a signal, enabling signal is input successively to the respective driver signal lines 151 to turn on the respective switch transistors 153 row by row, and in this manner, each detection signal line 152 receives a photocurrent signal generated by a photosensitive detection electrode 141. Alternatively enabling signal is input to a plurality of driver signal lines 151 simultaneously to turn on a plurality of switch transistors 153 concurrently, and in this manner each detection signal line 152 receives the sum of photocurrent signals generated by a plurality of photosensitive detection electrodes 141. Alike the enabling signal is input to all the driver signal lines 151 so that each detection signal line 152 receives photocurrent signals generated by a column of photosensitive detection electrodes 141. In a specific implementation, a driving mode of the driver signal lines 151 is determined according to the strength of the light source for heart rate detection. For example, when the strength of the light emitted from the light source is high, enabling signal is input successively to the respective driver signal lines 151, and in this manner, a detection signal line 152 reads a photocurrent signal of a photosensitive detection electrode 141 to thereby obtain heart rate information, without inputting enabling signal to all the driver signal lines 151; and when the strength of the light emitted from the light source is low, enabling signal is input to a plurality of driver signal lines 151, and at most all the driver signal lines 151 simultaneously, so that a detection signal line 152 reads the sum of photocurrent signals of a plurality of photosensitive detection electrodes 141 to thereby enhance a photocurrent signal read by the detection signal line 152 so as to detect heart rate information accurately despite of weak light.

Further referring to FIG. 2B and FIG. 2C, in the structure of the photosensitive layer 14 in another pattern, when detecting a signal, enabling signal is input successively to the respective driver signal lines 151, and in this manner, each detection signal line 152 reads a photocurrent signal of a strip-shaped photosensitive detection electrode 141; or enabling signal is input to a plurality of driver signal lines 151 simultaneously so that each detection signal line 152 reads the sum of photocurrent signals of a plurality of strip-shaped photosensitive detection electrodes 141 to thereby increase the strength of a signal obtained by the detection signal line 152 so as to facilitate detection of heart rate information. Alike a driving mode of the driver signal lines 151 is determined according to the strength of the light source for heart rate detection, and the more the driver signal lines to which enabling signal is input simultaneously are, the higher the strength of the a photocurrent signal obtained by the detection signal line 152 is, thus further facilitating heart rate detection, so the number of driver signal lines 151 to be driven simultaneously is determined according to the strength of the light source.

Further referring to FIG. 2D, since the photosensitive detection electrode 141 is arranged as an integral plane, in this manner the driver signal lines 151 and the switch transistors 153 connected with the driver signal lines 151 are not arranged, but only one detection signal line 152 is arranged, and when detecting a heart rate, a photocurrent signal received on the detection signal line 152 is obtained directly.

In some embodiments of the disclosure, in the display device above, as illustrated in FIG. 4, the photosensitive detection circuit further includes: a current-voltage conversion circuit 154, a filter circuit 155 connected with the current-voltage conversion circuit 154, an amplifying circuit 156 connected with the filter circuit 155, a counter 157 connected with the amplifying circuit 156, and a timer 158 connected with the counter 157.

The current-voltage conversion circuit 154 is connected with the detection signal line 152, and configured to convert a photocurrent signal detected on the detection signal line 152 into a voltage signal.

The filter circuit 155 is configured to filter the voltage signal.

The amplifying circuit 156 is configured to amplify the filtered voltage signal.

The counter 157 is configured to count under the control of the timer to obtain the value of a heart rate.

When the light reflected by the skin is incident onto a photosensitive detection electrode 141, the light is received by the photosensitive detection electrode 141 to generate a photocurrent signal, the photocurrent signal is transmitted to the current-voltage conversion circuit through the detection signal line 152, the current-voltage conversion circuit converts the photocurrent signal into a voltage signal, then the filter circuit filters the voltage signal, for example, through band-pass filtering, to remove low-frequency noise and high-frequency noise, furthermore the amplifying circuit amplifies the filtered voltage signal into an alternating-current signal with appropriate amplitude, and the value of the heart rate is obtained by counter counting. In a real application, when detecting heart rate information, the photosensitive detection circuit inputs a start signal (for example, Start Clk) to the timer to start the timer, the timer starts the counter into counting, and outputs the value of the heart rate at the end of timing, so that the obtained value of the heart rate is displayed by the organic light-emitting display element 12.

In some embodiments of the disclosure, in the display device above, the driver signal line 151 is further connected with the organic light-emitting display element 12 to drive the organic light-emitting display element 12 to display an image in a display period, and to drive the photosensitive detection electrode 141 to generate a photocurrent signal and transmit to the photosensitive detection circuit in a heart rate detection period.

In some embodiments of the disclosure, the driver signal line 151 operates in a time-division multiplexing mode to drive the photosensitive detection electrode 141 to generate a photocurrent signal and transmit to the photosensitive detection circuit, and to drive the organic light-emitting display element 12 to display an image, thus the number of driver signal lines 151 is reduced, and the image displaying and the heart rate detection do not influence each other, that is, a heart rate can be detected while an image is being displayed.

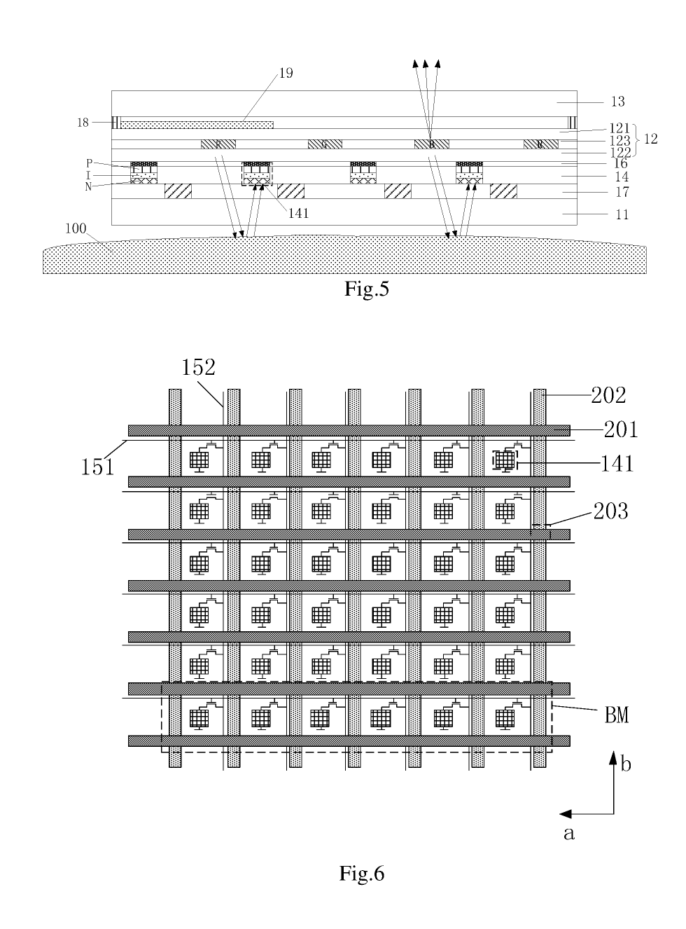

In some embodiments of the disclosure, in the display device above, as illustrated in FIG. 5 and FIG. 6, the display device further includes a black matrix layer 19 located on the side of the organic light-emitting display element away from the base substrate 11. As illustrated in FIG. 5, the black matrix layer 19 may be arranged between the first electrode layer 121 and the encapsulation layer 13, that is, the black matrix layer 19 is located on the layer where the frame sealant 18 is located.

The black matrix layer 19 covers a portion of the organic light-emitting display element 12 (in the area denoted by BM as illustrated in FIG. 6), and covers at least one photosensitive detection electrode 141.

The photosensitive detection circuit further includes a mode switching circuit.

The mode switching circuit is configured, in an active light mode, to control the photosensitive detection electrodes 141 in the area covered by the black matrix layer 19 to detect the first light reflected by the skin and generate a photocurrent signal, and in an ambient light mode, to control the photosensitive detection electrodes 141 out of the area covered by the black matrix layer 19 to detect the ambient light reflected by the skin and generate a photocurrent signal.

Referring to FIG. 5, the black matrix layer 19 covers a portion of the organic light-emitting display element 12, and covers at least one photosensitive detection electrode 141, while an image is being displayed by such a portion of the organic light-emitting display element 12 that are not shielded by the black matrix layer 19, the light-emitting elements 203 covered by the black matrix layer 19 can be used as a light source, and the photosensitive detection electrode 141 covered by the black matrix layer can detect the first light reflected by the skin and generate a photocurrent signal to detect heart rate information, so that the image can be displayed while a heart rate is being detected, instead of operating in a time-division multiplexing mode.

While the organic light-emitting display element 12 does not emit light, ambient light can be used as a light source for heart rate detection. The ambient light is transmitted to the skin through the display device, and reflected by the skin, and then the reflected ambient light is transmitted to the photosensitive detection electrodes 141, and the photosensitive detection electrodes 141 which are not shielded by the black matrix layer 19 are controlled to detect the ambient light reflected by the skin and generate a photocurrent signal, so that a heart rate can be detected while no image is being displayed by the display element.

Specifically the active light mode is a period in which the organic light-emitting display element is displaying an image or sleeping, and the ambient light mode is a period in which the organic light-emitting display element is disabled.

In a real application, in some application scenario, a heart rate still should be detected while the display screen is sleeping or on standby, and at this time, the light-emitting elements which are not shielded by the black matrix layer do not emit light; and in order to detect a heart rate, only the light-emitting elements located below the black matrix layer are controlled to provide a light source for heart rate detection, and the photosensitive detection electrodes located below the black matrix layer are driven to detect the first light and generate a photocurrent signal to thereby detect a heart rate. Furthermore when the strength of the ambient light is high, all the light-emitting elements are disabled, the ambient light is used directly as a light source for heart rate detection, and the photosensitive detection electrodes which are not shielded by the black matrix layer are driven to detect the ambient light and generate a photocurrent signal to thereby detect a heart rate. Specifically a mode switching circuit is arranged to enable the organic light-emitting display element to detect a heart rate in the active light mode and the ambient light mode.

As illustrated in FIG. 1, in the display device above according to the embodiment of the disclosure, the photosensitive layer 14 is located on the side of the organic light-emitting display element 12 close to the base substrate 11, so that there is such a short distance between the photosensitive layer 14 and the base substrate 11, i.e., such a short distance between the photosensitive layer 14 and the skin of a user, that it will be easier for the light reflected by the skin to be transmitted to the photosensitive layer 14 through the display device.

In some embodiments of the disclosure, the display device further includes a line layer 17 located between the base substrate 11 and the organic light-emitting display element 12.

The photosensitive detection circuit is arranged at the same layer as the line layer 17.

The photosensitive layer 14 is located between the organic light-emitting display element 12 and the line layer 17.

In a specific implementation, the line layer 17 includes a driver circuit configured to drive the organic light-emitting display element 12, and the photosensitive detection circuit is arranged at the same layer as the line layer 17 so that the photosensitive detection circuit is added without increasing the volume of the display device.

The photosensitive layer 14 is arranged between the organic light-emitting display element 12 and the line layer 17 so that there is such a distance between the photosensitive layer 14 and the line layer 17 that it will be easy for a photocurrent signal generated by the photosensitive layer 14 to be transmitted to the photosensitive detection circuit. Furthermore there is such a short distance between the photosensitive layer 14 and the base substrate 11 that it will be easy for the light reflected by the skin to be transmitted to the photosensitive layer 14 while a heart rate is being detected. The photosensitive layer 14 is optionally arranged between the organic light-emitting display element 12 and the line layer 17, but in a specific implementation, the photosensitive layer 14 can alternatively be arranged at another position, e.g., between the line layer 17 and the base substrate 11, although the position of the photosensitive layer will not be limited thereto.

Specifically in the display device above according to the embodiment of the disclosure, the photosensitive detection electrode is made of a PIN-type semiconductor material or a PN-type semiconductor material. As illustrated in FIG. 1, when the photosensitive detection electrode 141 is made of a PIN-type semiconductor material, P, I and N areas of the PIN-type semiconductor material are optionally stacked over each other. Similarly when the photosensitive detection electrode 141 is made of a PN-type semiconductor material, P and N areas of the PN-type semiconductor material are optionally stacked over each other.

In another aspect, based upon the same inventive idea, embodiments of the disclosure further provide a method of detecting heart rate information by the display device above. Since the method addresses the problem under a similar principle to the display device above, reference can be made to the implementation of the display device above for an implementation of the method, and a repeated description thereof will be omitted here.

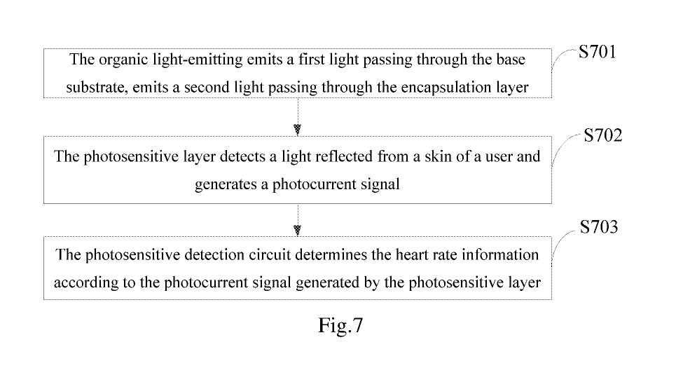

As illustrated in FIG. 7, the method includes the following steps.

In the step S701, the organic light-emitting emits a first light passing through the base substrate, emits a second light passing through the encapsulation layer.

In the step S702, the photosensitive layer detects a light reflected from a skin of a user and generates a photocurrent signal.

In the step S703, the photosensitive detection circuit determines the heart rate information according to the photocurrent signal generated by the photosensitive layer.

In some embodiments, the photosensitive layer detects the first light reflected from the skin of the user and/or the ambient light reflected from the skin of the user, and generates a photocurrent signal.

In the display device according to the embodiment of the disclosure, the organic light-emitting display element emits light bidirectionally, and the photosensitive layer is arranged between the base substrate and the encapsulation layer, so that the photosensitive layer can use the light emitted from the organic light-emitting display element, or the ambient light as a light source, without arranging light source separately, thus reducing the size of a heart rate detection element, and lowering power consumption of heart rate detection.

Evidently those skilled in the art can make various modifications and variations to the disclosure without departing from the spirit and scope of the disclosure. Thus the disclosure is also intended to encompass these modifications and variations thereto so long as the modifications and variations come into the scope of the claims appended to the disclosure and their equivalents.

1. A display device, comprising:
a base substrate;
an encapsulation layer, on the base substrate;
an organic light-emitting display element between the base substrate and the encapsulation layer configured to emit a first light passing though the base substrate and a second light passing through the encapsulation layer to display image;
a photosensitive layer between the base substrate and the encapsulation layer configured to detect light reflected from a skin of a user and generate a photocurrent signal; and a photosensitive detection circuit coupled to the photosensitive layer configured to determine heart rate information according to the photocurrent signal generated by the photosensitive layer and output the heart rate information.

2. The display device according to claim 1, further comprises a light-shielding layer located on a side of the photosensitive layer away from the base substrate, wherein:
a positive projection of the light-shielding layer onto the base substrate overlaps with a positive projection of the photosensitive layer onto the base substrate.

3. The display device according to claim 1, wherein the organic light-emitting display element comprises: a first electrode layer, a second electrode layer located on a side of the first electrode layer close to the base substrate, and an organic functional layer located between the first electrode layer and the second electrode layer, wherein:
the first electrode layer and the second electrode layer are made of a transparent electrically-conductive material.

4. The display device according to claim 1, wherein the photosensitive layer comprises at least one photosensitive detection electrode coupled to the photosensitive detection circuit, the at least one photosensitive detection electrode is configured to detect the light reflected from the skin of the user and generate the photocurrent signal.

5. The display device according to claim 4, wherein the first electrode layer comprises a plurality of first electrodes extending along a first direction;
the second electrode layer comprises a plurality of second electrodes extending along a second direction intersecting with the first direction;
the organic functional layer comprises light-emitting elements at positions where the first electrodes intersect with the second electrodes;
a positive projection of the at least one photosensitive detection electrode does not fully overlap with positive projections of the light-emitting elements onto the base substrate.

6. The display device according to claim 5, wherein the photosensitive layer comprises a plurality of photosensitive detection electrodes distributed in an array, wherein:
positive projections of the photosensitive detection electrodes onto the base substrate do not overlap with any of positive projections of the first electrodes, the second electrodes, and the light-emitting elements onto the base substrate.

7. The display device according to claim 5, wherein the photosensitive layer comprises a plurality of strip-shaped photosensitive detection electrodes arranged in parallel, wherein:
the photosensitive detection electrodes extend along the first direction, and positive projections of the photosensitive detection electrodes and the positive projections of the first electrodes onto the base substrate do not overlap with each other; or
the photosensitive detection electrodes extend along the second direction, and the positive projections of the photosensitive detection electrodes and the positive projections of the second electrodes onto the base substrate do not overlap with each other.

8. The display device according to claim 5, wherein the photosensitive layer comprises a photosensitive detection electrode arranged as an integral plane, wherein:
the photosensitive detection electrode is provided with hollow areas at its portions overlapping with the light-emitting elements.

9. The display device according to claim 5, wherein the photosensitive detection circuit comprises: at least one driver signal line extending along the first direction, at least one detection signal line extending along the second direction, and at least one switch transistor, wherein:
the switch transistor has a gate connected with the driver signal line, a source connected with the photosensitive detection electrode, and a drain connected with the detection signal line.

10. The display device according to claim 9, wherein the photosensitive detection circuit further comprises: a current-voltage conversion circuit, a filter circuit connected with the current-voltage conversion circuit, an amplifying circuit connected with the filter circuit, a counter connected with the amplifying circuit, and a timer connected with the counter, wherein:
the current-voltage conversion circuit is connected with the at least one detection signal line, and configured to convert the photocurrent signal on the at least one detection signal line into a voltage signal;
the filter circuit is configured to filter the voltage signal;
the amplification circuit is configured to amplify the filtered voltage signal; and
the counter is configured to count under the control of the timer to obtain a value of a heart rate.

11. The display device according to claim 9, wherein the at least one driver signal line is further connected with the organic light-emitting display element to drive the organic light-emitting display element to display the image in a display period, and to drive the photosensitive detection electrode to generate the photocurrent signal and transmit the photocurrent signal to the photosensitive detection circuit in a heart rate detection period.

12. The display device according to claim 9, further comprises a black matrix layer located on a side of the organic light-emitting display element away from the base substrate;
the black matrix layer covers a portion of the organic light-emitting display element, and covers at least one photosensitive detection electrode; and
the photosensitive detection circuit further comprises a mode switching circuit, wherein:
the mode switching circuit is configured, in an active light mode, to control the photosensitive detection electrodes in an area covered by the black matrix layer to detect the first light reflected from the skin and generate the photocurrent signal, and in an ambient light mode, to control the photosensitive detection electrodes out of the area covered by the black matrix layer to detect an ambient light reflected from the skin and generate the photocurrent signal.

13. The display device according to claim 12, wherein the active light mode is a period when the organic light-emitting display element is displaying an image or sleeping, and the ambient light mode is a period when the organic light-emitting display element is disabled.

14. The display device according to claim 1, wherein the photosensitive layer is configured to detect the first light and/or an ambient light reflected from the skin of the user, and generate the photocurrent signal.

15. The display device according to claim 1, wherein the photosensitive layer is located on a side of the organic light-emitting display element close to the base substrate.

16. The display device according to claim 15, further comprises a line layer located between the base substrate and the organic light-emitting display element, wherein:
the photosensitive detection circuit is arranged at the same layer as the line layer; and
the photosensitive layer is located between the organic light-emitting display element and the line layer.

17. The display device according to claim 1, wherein the display device is a wearable display device.

18. The display device according to claim 1, wherein the photosensitive detection electrode is made of a PIN-type semiconductor material or a PN-type semiconductor material.

19. A method of detecting heart rate information by the display device according to claim 1, the method comprises:
emitting, by the organic light-emitting display element, a first light passing through the base substrate, emitting a second light passing through the encapsulation layer;
detecting, by the photosensitive layer, light reflected from a skin of a user and generating a photocurrent signal; and
determining, by the photosensitive detection circuit, the heart rate information according to the photocurrent signal generated by the photosensitive layer.

20. The method according to claim 19, wherein detecting, by the photosensitive layer, the light reflected from the skin of the user and generating the photocurrent signal comprises:
detecting, by the photosensitive layer, the first light and/or ambient light reflected from the skin of the user, and generating the photocurrent signal.
